(19) United States
(12) Patent Application Publication
(10) Pub. No.: US 2013/0145527 A1
(43) Pub. Date: Jun. 13, 2013

(54) PROTECTIVE GLASSES

(71) Applicants: Yoshihiro Miwa, Nagoya-shi (JP); Hirofumi Tominaga, Higashiosaka-shi (JP); Masataka Asada, Higashiosaka-shi (JP)

(72) Inventors: Yoshihiro Miwa, Nagoya-shi (JP); Hirofumi Tominaga, Higashiosaka-shi (JP); Masataka Asada, Higashiosaka-shi (JP)

(73) Assignee: KOWA COMPANY, LTD., Aichi (JP)

(21) Appl. No.: 13/699,145

(22) PCT Filed: Aug. 26, 2011
(86) PCT No.: PCT/JP2011/069245

§ 371 Date: Feb. 26, 2013

(30) Foreign Application Priority Data
Aug. 26, 2010 (JP) 2010-189805

(51) Int. Cl. A61F 9/02

(52) U.S. Cl. CPC A61F 9/02
USPC 2/431

(57) ABSTRACT

Protective glasses include a brace bar, arms attached to both ends of the brace bar, and a lens wherein a central upper part of the lens is fixedly attached to a center part of the brace bar. A canopy part having a shape corresponding to the face of a wearer protrudes from the peripheral edge of the lens. The lens is designed so as not to deform even if the wearer has a large head, and thus the gap between the canopy part and the face of the wearer does not increase. Therefore, the eyes of the wearer can be protected while ingress of matter such as pollen and dust is effectively prevented. In addition, since the angle of the optical axis does not change, images seen through the lens are not distorted.

### TECHNICAL FIELD

The invention relates to protective glasses for protecting eyes from pollen, dust and the like.

### BACKGROUND ART

Conventionally, as glasses of this kind, glasses in which lenses are fitted in frames with canopy parts, glasses in which lenses with canopy parts are fitted in frames, glasses in which canopy parts are integrated with single lenses and the like exist, as disclosed in Patent Literatures 1 to 4.

In the above described glasses of this kind, in the glasses in which a canopy part is integrated with a single lens, a space between left and right arms 12 and 12 which are attached to both end portions of a single lens 11 is formed to be an ideal head size that is estimated in design as shown in, for example, FIG. 11, and therefore, when a wearer has the ideal head size, a canopy part 13 which is integrally provided at an upper edge of the lens 11 can cover a face surface of the wearer without generating a large gap S between the canopy part and the face surface of the wearer, as shown in FIG. 12.

Meanwhile, apart from the glasses as described above, there exist glasses in which the images seen through the lens are less affected by the dimension of the head size (face width), as disclosed in Patent Literature 5.

The glasses include a brace bar 21 and a nose pad portion 22, have arms 23 attached to both end portions of the brace bar 21, and hold a single lens 24 with a central portion of the aforementioned brace bar 21 and the nose pad portion 22. Further, left and right upper edge portions of the aforementioned single lens 24 are disposed forward by being provided with a space g with respect to the brace bar 21 as shown in FIGS. 14 and 15. The space between the aforementioned left and right arms 23 and 23 is formed to be an ideal head size which is estimated in design.

Therefore, in the above described conventional glasses, when a head size (face width) is large, the left and the right arms 23 and 23 are opened from each other, and the brace bar 21 is elastically deformed. However, the glasses hold only a central portion of the single lens 24 with the central portion of the brace bar 21 and the nose pad portion 22, and therefore, the single lens 24 is deformed in only the portion which does not have an influence on the field of view, whereby the image which is seen through the single lens 24 is hardly distorted.

### Citation List

### Patent Literatures

### Patent Literature 1: Japanese Patent Laid-Open No. 10-328230

### Patent Literature 2: Japanese Utility Model Laid-Open No. 5-60890

### Patent Literature 3: Registered Japanese Utility Model No. 3026228

### Patent Literature 4: Registered Japanese Utility Model No. 3152412

### Patent Literature 5: Japanese Patent Laid-Open No. 2002-214567

### SUMMARY OF INVENTION

### Technical Problem

However, in the conventional protective glasses shown in FIG. 11 described above, when the head size of a wearer is larger than the ideal head size which is estimated in design, the left and the right arms 12 and 12 are forced to be opened by the temporal region of the wearer as shown in FIG. 13. The left and the right arms 12 and 12 are attached to both the end portions of the single lens 11 in this case, and therefore, if these left and right arms 12 and 12 are forced to be opened, the entire single lens 11 is also elastically deformed naturally, whereby the following problems arise.

First, the optical axis angle of the single lens 11 changes, and distortion occurs to the image which is seen through the single lens 11. Subsequently, the gap S between the canopy part 13 of the single lens 11 and the face surface of the wearer becomes large, and pollen, dust and the like cannot be efficiently prevented from entering the eyes. Furthermore, the single lens 11 is formed from a rigid synthetic resin or the like and has elasticity, and therefore, when the single lens 11 is elastically deformed, the temporal regions of the wearer are pressed by the left and the right arms 12 and 12.

Further, in the above described conventional glasses shown in FIG. 14, in the single lens 24, only the portion which does not have an influence on the field of view is deformed even when the head size (face width) of the wearer is large. Therefore, the image which is seen through the single lens 24 is hardly distorted, but the gap between the single lens 24 and the face surface of the wearer is widely opened, and pollen, dust and the like can be hardly prevented from entering the eyes.

Therefore, this invention has an object to solve the above described conventional problems, and this invention is made with an object to provide protective glasses which can protect eyes by efficiently preventing pollen, dust and the like from entering eyes without increasing a gap between a canopy part and a face surface of a wearer by preventing a lens from being deformed even when a head size of the wearer is large, and does not change an optical axis angle by further preventing the lens from being deformed to prevent an image seen through the lens from being distorted.

### Solution to Problem

Protective glasses of this invention comprises a brace bar, arms attached to both ends of the brace bar, and a lens, wherein a central upper part of the lens is fixedly attached to a center part of the brace bar, and a canopy part having a shape corresponding to the face of a wearer protrudes from the peripheral edge of the lens.

In the protective glasses of this invention, the brace bar is formed from a synthetic resin which is elastically deformable.

Further, in the protective glasses of this invention, the lens is a single lens, has a laterally long length substantially corresponding to a length of the brace bar, and is formed into a substantially arc shape in plan view so that an upper edge is substantially along the brace bar.

Further, in the protective glasses of this invention, the lens is a twin-lens, with respective inner upper portions being connected to each other with a bridge portion, has a laterally long length substantially corresponding to a length of the brace bar, and is formed into a substantially arc shape in plan view so that an upper edge is substantially along the brace bar.

Furthermore, in the protective glasses of this invention, the canopy part is protruded to a face surface direction of the wearer to be along a forehead lower portion of the face surface of the wearer.

Further, in the protective glasses of this invention, the canopy part is protruded to the face surface direction of the wearer to be along a cheek upper portion and vicinities of temples of the face surface of the wearer.

### Advantageous Effects of Invention

Since the protective glasses of this invention is configured as described above, the protective glasses can protect eyes by efficiently preventing pollen, dust and the like from entering eyes without increasing a gap between a canopy part and a face surface of a wearer even when a head size of the wearer is large, and further, does not change an optical axis angle to prevent an image seen through the lens from being distorted.

### BRIEF DESCRIPTION OF DRAWINGS

FIG. 1 is a perspective view showing one embodiment of protective glasses of this invention.

FIG. 2 is an exploded perspective view of the protective glasses of this invention shown in FIG. 1.

FIG. 3 is a plan view showing a state in which a nose pad of the protective glasses of this invention shown in FIG. 1 is removed.

FIG. 4 is a rear view of the protective glasses of this invention shown in FIG. 1.

FIG. 5 is a cross-sectional view of the protective glasses of this invention taken along the A-A line in FIG. 4.

FIG. 6 is a perspective view showing another embodiment of the protective glasses of this invention.

FIG. 7 is an exploded perspective view of the protective glasses of this invention shown in FIG. 6.

FIG. 8 is a plan view of the protective glasses of this invention shown in FIG. 6.

FIG. 9 is a plan view showing a state in which the protective glasses of this invention shown in FIG. 1 are worn by a user with an ideal head size estimated in design.

FIG. 10 is a plan view showing a state in which the protective glasses of this invention shown in FIG. 1 are worn by a user with a head size larger than the ideal head size estimated in design.

FIG. 11 is a perspective view showing one example of conventional protective glasses.

FIG. 12 is a plan view showing a state in which the conventional protective glasses shown in FIG. 11 are worn by the user with the ideal head size estimated in design.

FIG. 13 is a plan view showing a state in which the conventional protective glasses shown in FIG. 11 are worn by the user with the head size larger than the ideal head size estimated in design.

FIG. 14 is a front view showing one example of the conventional glasses which prevent the image seen through the lens from being distorted.

FIG. 15 is a plan view showing the conventional glasses shown in FIG. 14 by being partially omitted.

### DESCRIPTION OF EMBODIMENTS

Hereinafter, the embodiments of protective glasses of this invention will be described in detail based on the drawings.

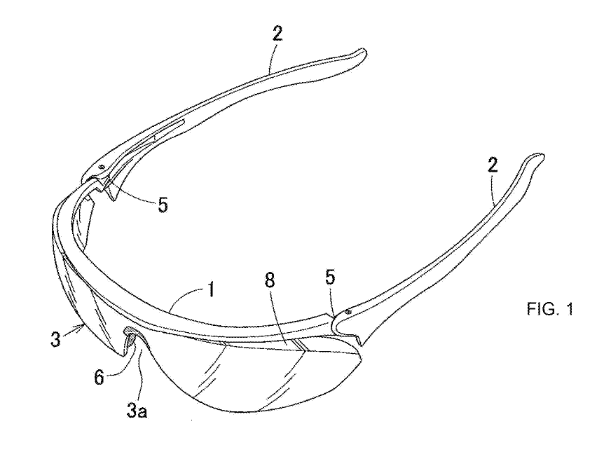

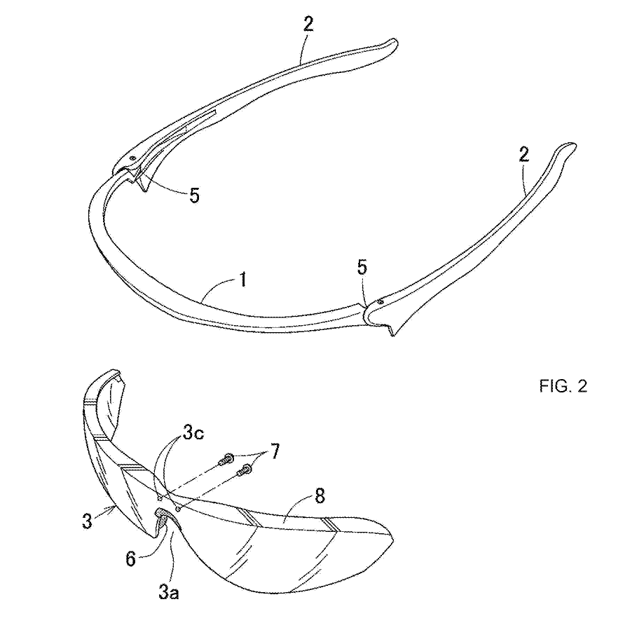

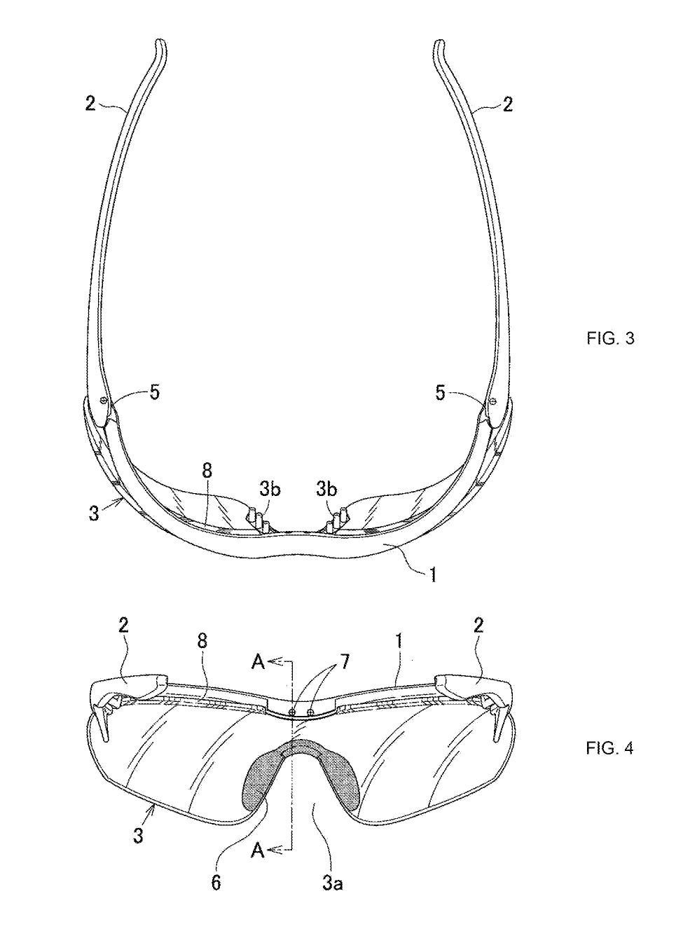

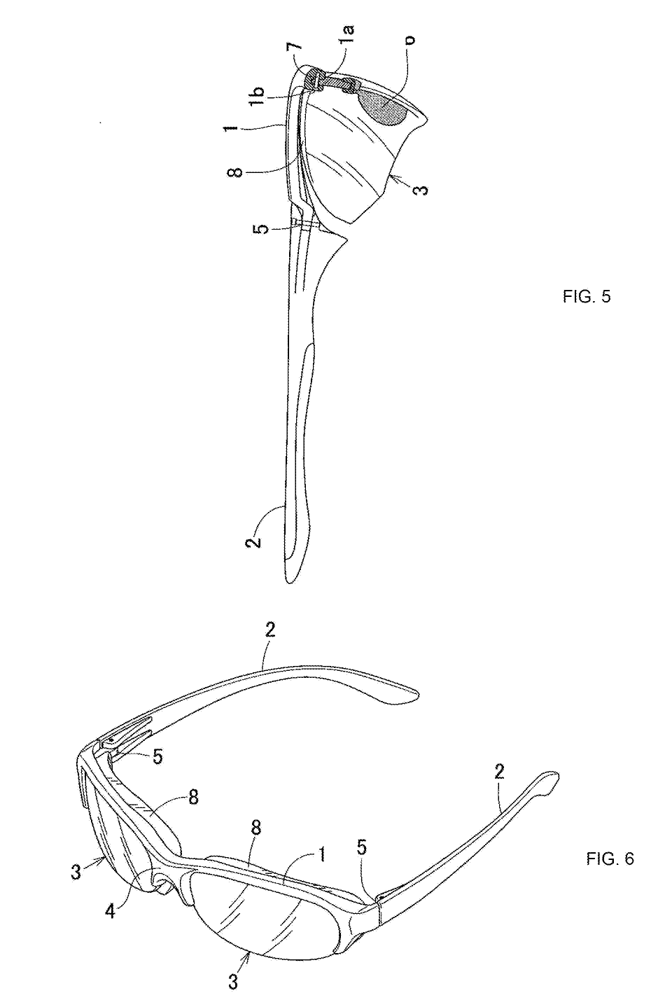

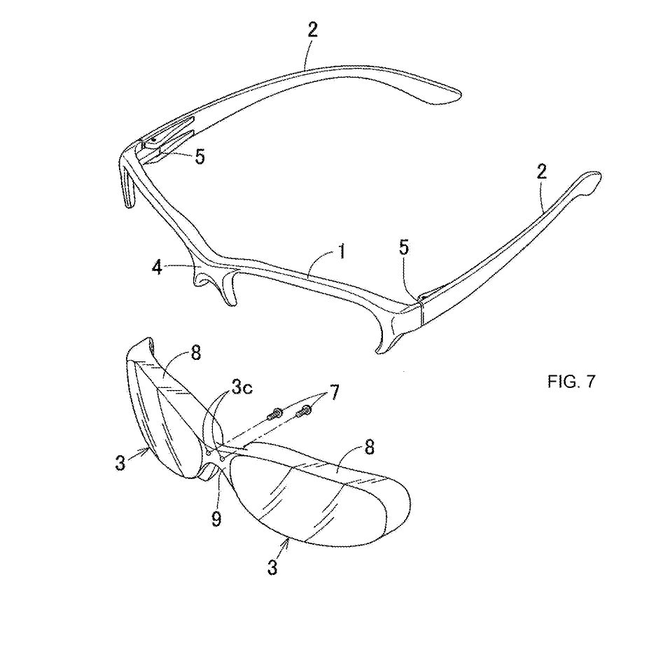

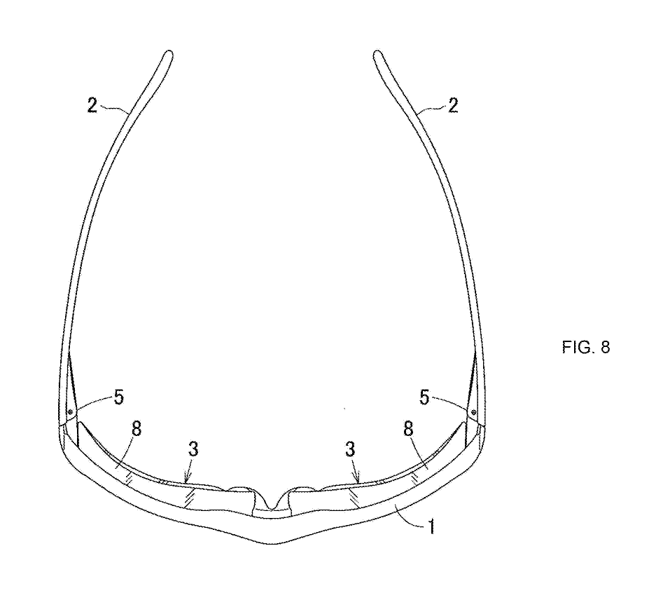

As for the protective glasses of this invention, the protective glasses shown in FIGS. 1 to 5 are constituted of a brace bar 1, arms 2 attached to both ends of the brace bar 1, and a single lens 3, and the protective glasses shown in FIGS. 6 to 8 are constituted of the brace bar 1 in which a bridge portion 4 is formed in a center, the arms 2 attached to both ends of the brace bar 1, and a twin-lens 3.

The brace bar 1 is formed from a synthetic resin elastically deformable such as nylon and polycarbonate, and the bridge portion 4 formed in the center is integrally molded with the brace bar 1. Further, the aforementioned brace bar 1 is formed into a substantially arc shape in plan view so as to be substantially along the shape of a forehead lower portion of a wearer with an ideal head size estimated in design.

The arms 2 are formed from the same synthetic resin as the aforementioned brace bar 1, and are attached to the brace bar 1 to be foldable via hinges 5.

The single lens 3 is formed from polycarbonate or the like which is more rigid than the aforementioned brace bar 1 and arms 2, has a laterally long length substantially corresponding to the length of the aforementioned brace bar 1, and is formed into a substantially arc shape in plan view so that an upper edge is substantially along the brace bar 1.

The aforementioned single lens 3 forms a cutout portion 3a in a central lower portion, and a nose pad 6 is attached to an inside of the cutout portion 3a. An engaging projected portion 3b for being attachably and detachably engaged in an engaging recessed portion (not illustrated) which is formed in the nose pad 6 is formed in the inside of the aforementioned cutout portion 3a. A central upper portion of the aforementioned lens 3 is fixed to a central portion of the brace bar 1 by being fastened with screws or the like. In the illustrated glasses, the central upper portion of the aforementioned lens 3 is fitted in a groove la which is provided in the central portion of the brace bar 1, screws 7 are inserted into two screw holes 3c which are provided on the left and the right in the central upper portion of the lens 3 at a space of 3 to 10 mm, and two screw holes 1b which are provided in the central portion of the brace bar 1 to oppose the screw holes 3c, respectively, and these screws 7 are screwed into the brace bar 1, whereby the aforementioned lens 3 is fastened to the brace bar 1 with the screws. The space between the aforementioned two screw holes 7 is desired to be as narrow as possible within the range without causing a problem in strength so that distortion of the brace bar 1 hardly influences the lens 3 when the aforementioned lens 3 is fixed to the brace bar 1. Fixation of the aforementioned lens 3 to the brace bar 1 can be performed similarly to the case of the twin-lens if the component corresponding to the bridge portion 4 of the twin-lens which will be described later is provided in the central portion of the brace bar 1, instead of providing the groove la in the central portion of the brace bar 1.

Furthermore, in the aforementioned lens 3, a canopy part 8 which is projected to be along the face surface of the wearer is formed at the edge thereof. Namely, the canopy part 8 which is projected to an inward direction (face surface direction of the wearer) is integrally molded at the upper edge of the aforementioned lens 3 to be along the forehead lower portion of the face surface of the wearer. Further, in the aforementioned lens 3, the canopy part 8 (not illustrated) which is projected to the inward direction (face surface direction of the wearer) can be integrally molded at a lower edge thereof to be along a cheek upper portion of the face surface of the wearer, or the canopy part 8 (not illustrated) which is projected to the inward direction (face surface direction of the wearer) can be integrally molded at both side edges thereof to be along the vicinities of the temples of the face surface of the wearer, in accordance with necessity.

The twin-lens 3 is formed from polycarbonate more rigid than the aforementioned brace bar 1 and the arms 2, inner upper portions thereof are connected to each other with a bridge portion 9, has a laterally long length substantially corresponding to the length of the aforementioned brace bar 1, and is formed into a substantially arc shape in plan view so that an upper edge is substantially along the brace bar 1. A central upper portion of the aforementioned lens 3, namely, the bridge portion 9 is fixed to a central portion of the brace bar 1, namely, the bridge portion 4 by being fastened with screws or the like. In the illustrated glasses, the screws 7 are respectively inserted into two screw holes 3c provided on the left and the right from the center of the bridge portion 9 of the aforementioned lens 3 at a space of 3 to 10 mm, and the screws 7 are screwed into the bridge portion 4, whereby the aforementioned lens 3 is fastened to the brace bar 1 with the screws. The space between the aforementioned two screw holes 7 is desired to be as narrow as possible within the range without causing a problem in strength so that distortion of the bridge portion 9 hardly influences the lens 3 when the aforementioned lens 3 is fixed to the brace bar 1. Further, the thickness and the width of the aforementioned bridge portion 4 are made larger than the brace bar 1, so that the variation is given to make the glasses excellent in design, and a problem in strength does not occur due to the manner of the fixation of the aforementioned lens 3 or the like. Fixation of the aforementioned lens 3 to the brace bar 1 can be also performed similarly to the case of the single lens if the component corresponding to the groove 1 a of the single lens described above is provided in the central portion of the brace bar 1, instead of providing the bridge portion 4 in the central portion of the brace bar 1.

Furthermore, in the aforementioned lens 3, the canopy part 8 which is protruded to be along the face surface of the wearer is formed at the edge thereof. Namely, the canopy part 8 which is protruded to the inward direction (face surface direction of the wearer) is provided at the entire edge of the aforementioned lens 3 so as to be along the forehead lower portion, the cheek upper portion and the vicinities of the temples of the face surface of the wearer. In the aforementioned lens 3, the canopy part 8 may be partially provided at an upper edge, a lower edge and both side edges thereof as in the aforementioned single lens 3 instead of providing the canopy part 8 at the entire edge.

The aforementioned canopy part 8 is translucent, but can be made opaque in accordance with necessity. The aforementioned canopy part 8 can contain an ultraviolet absorber. In this case, if the aforementioned lens 3 is made to contain an ultraviolet absorber, the canopy part 8 which is integrally molded can be made to contain the ultraviolet absorber. Further, if the aforementioned lens 3 is formed from a material with a high light transmittance, the canopy part 8 which is integrally molded is also formed from the material with a high light transmittance, and if the aforementioned lens 3 is formed from a material with a low light transmittance conversely, the canopy part 8 which is integrally molded is also formed from the material with a low light transmittance.

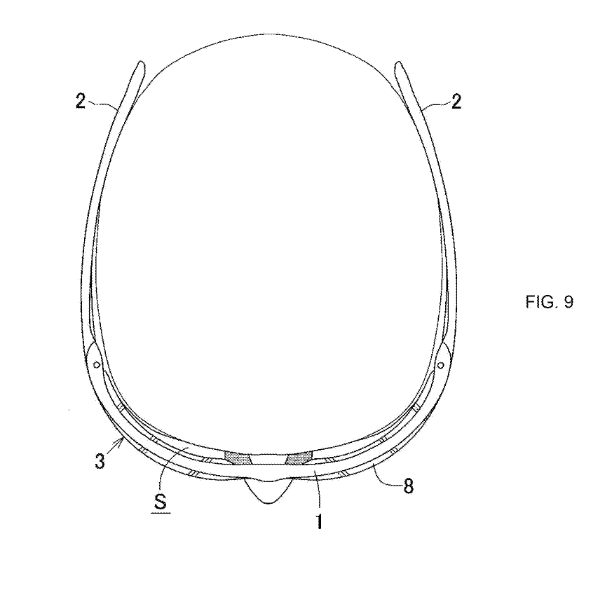

The protective glasses of this invention which is configured as above can efficiently prevent pollen, dust and the like from entering eyes in the case of the wearer with an ideal head size estimated in design, as shown in FIG. 9, because the brace bar 1 is not deformed at the time of being worn, and the lens 3 is not deformed, whereby the canopy part 8 provided at the edge of the lens 3 is also along the forehead lower portion, the cheek upper portion and the vicinities of the temples of the wearer, and the large gap S does not occur between these spots and the canopy part 8. The aforementioned lens 3 is not deformed, and therefore, distortion does not occur to the image which is seen through the lens 3. Furthermore, the aforementioned brace bar 1 is not deformed, and therefore, the temporal regions of the wearer are not pressed by the arms 2.

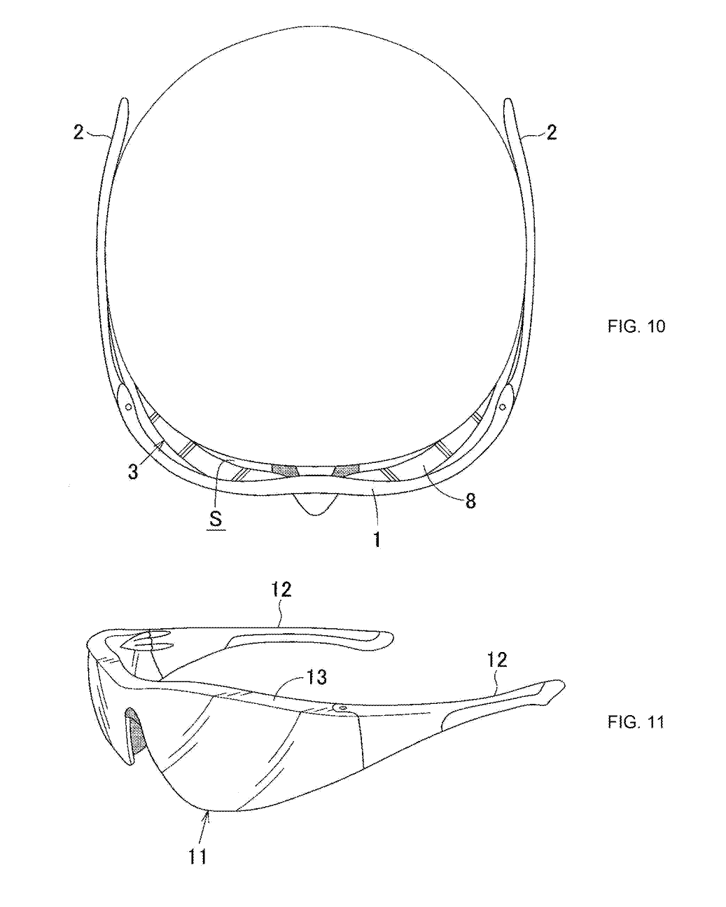

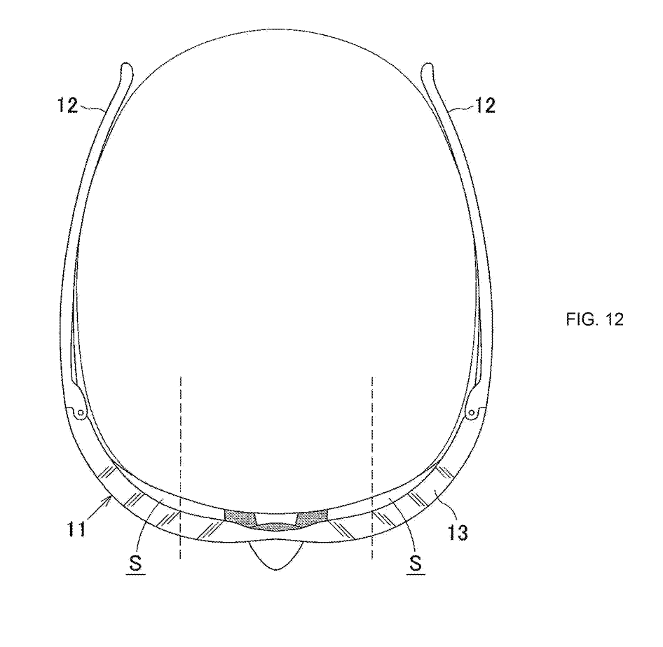

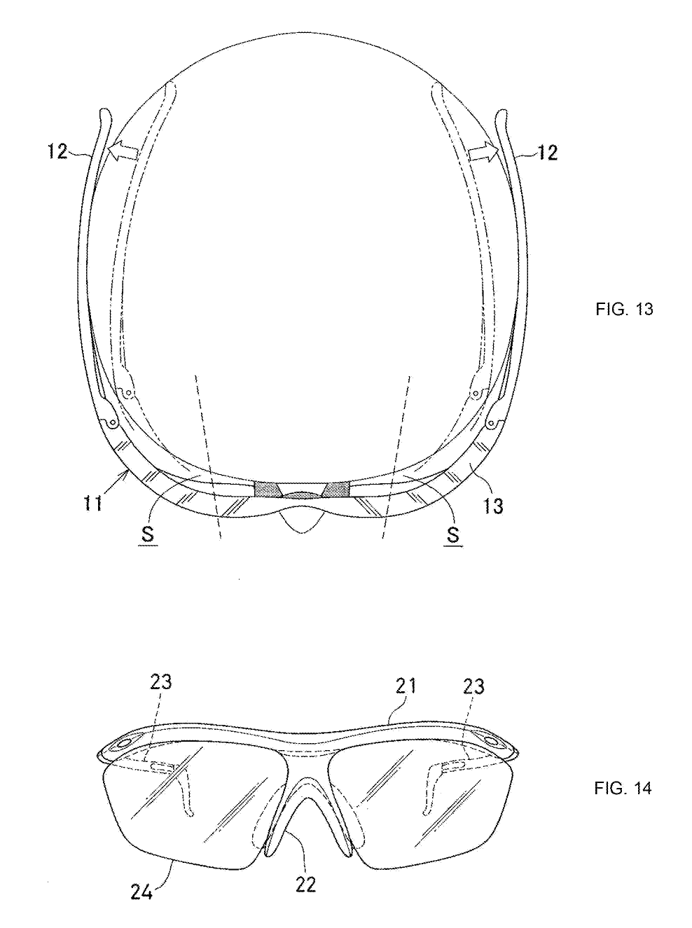

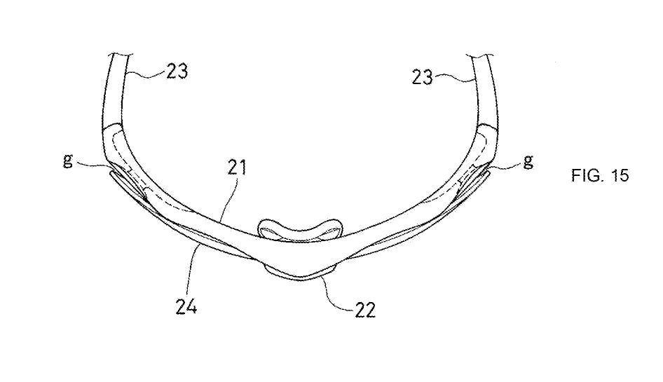

Furthermore, as for the protective glasses of this invention, in the case of the wearer with the head size larger than the ideal head size estimated in design, both the end portions of the brace bar 1 are deformed at the time of being worn, but the vicinity of the central portion of the brace bar 1 is hardly deformed as shown in FIG. 10, and therefore, the lens 3 which is fixed to the vicinity of the central portion of the brace bar 1 is not so deformed as to influence the visibility and the gap between the canopy part 8 and the face surface of the wearer. Accordingly, a large gap occurs between the brace bar 1 and the forehead lower portion of the wearer, but the canopy part 8 provided at the edge of the lens 3 is along the forehead lower portion, the cheek upper portion and the vicinities of the temples of the wearer, the large gap S does not occur between these spots and the canopy part 8, and entry of pollen, dust and the like into the eyes can be efficiently prevented. The aforementioned lens 3 is not deformed, and therefore, distortion does not occur to the image which is seen through the lens 3. Furthermore, the aforementioned brace bar 1 is formed from a synthetic resin elastically deformable, and therefore, even if both the end portions thereof are deformed, the temporal regions of the wearer are not pressed with the arms 2.

Further, when the canopy part 8 contains an ultraviolet absorber, the protective glasses of this invention can effectively prevent entry of ultraviolet rays from the side surfaces at the time of wearing the glasses, and furthermore, in the case when the canopy part 8 is formed with a material with a low light transmittance, eyes can be protected from strong sunlight from the side surfaces at the time of wearing the glasses.

1. Protective glasses comprising: a brace; arms attached to both ends of the brace bar; and a lens,
wherein a central upper part of the lens is fixedly attached to a center part of the brace bar, and
a canopy part having a shape corresponding to the face of a wearer protrudes from a peripheral edge of the lens.

2. The protective glasses according to claim 1,
wherein the brace bar is formed from a synthetic resin which is elastically deformable.

3. The protective glasses according to claim 1,
wherein the lens is a single lens, has a laterally long length substantially corresponding to a length of the brace bar, and is formed into a substantially arc shape in plan view so that an upper edge of the lens is substantially along the brace bar.

4. The protective glasses according to claim 1,
wherein the lens is a twin-lens, with respective inner upper portions being connected to each other with a bridge portion, has a laterally long length substantially corresponding to a length of the brace bar, and is formed into a substantially arc shape in plan view so that an upper edge of the lens is substantially along the brace bar.

5. The protective glasses according to claim 1,
wherein the canopy part is protruded to a face surface direction of the wearer to be along a forehead lower portion of a face surface of the wearer.

6. The protective glasses according to claim 3,
wherein the canopy part is protruded to a face surface direction of the wearer to be along a forehead lower portion of a face surface of the wearer.

7. The protective glasses according to claim 4,
wherein the canopy part (8) is protruded to a face surface direction of the wearer to be along a forehead lower portion of the face surface of the wearer.

8. The protective glasses according to claim 5,
wherein the canopy part is protruded to the face surface direction of the wearer to be along a cheek upper portion and vicinities of temples of the face surface of the wearer.

9. The protective glasses according to claim 6,
wherein the canopy part is protruded to the face surface direction of the wearer to be along a cheek upper portion and vicinities of temples of the face surface of the wearer.

10. The protective glasses according to claim 7,
wherein the canopy part is protruded to the face surface direction of the wearer to be along a cheek upper portion and vicinities of temples of the face surface of the wearer.
